(19) United States
(12) Patent Application Publication
(10) Pub. No.: US 2022/0332669 A1
(43) Pub. Date: Oct. 20, 2022

(54) PROCESS FOR PREPARING BIPHENYL-2,2'-DIOLS

(71) Applicant: EVONIK OPERATIONS GMBH, Essen (DE)

(72) Inventors: Dirk FRIDAG, Haltern am See (DE); Anna Chiara SALE, Recklinghausen (DE); Robert FRANKE, Marl (DE); Alexander BR.A.CHER, Haltern am See (DE); Johannes KNOSSALLA, Gahlen (DE)

(73) Assignee: EVONIK OPERATIONS GMBH, Essen (DE)

(21) Appl. No.: 17/718,514

(22) Filed: Apr. 12, 2022

(30) Foreign Application Priority Data
Apr. 16, 2021 (EP) 21168829.6

(51) Int. Cl. C07C 29/32

(52) U.S. Cl. CPC C07C 29/32

(57) ABSTRACT

Process for preparing biphenyl-2,2′-diols.

The invention relates to a process for preparing biphenyl-2,2′-diols. The phenyl rings here may also have further substituents in addition to the OH groups.

EP 2906571 B1 describes on pages 11-12 the synthesis of 2,2′-bis(3,5-dimethylphenol) (1).

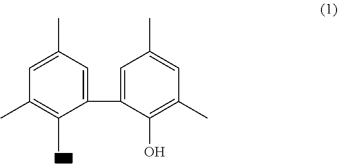

Here, 2,4-dimethylphenol is reacted with addition of FeSO₄ and Na₂S₂O₈. The product (1) is obtained with a yield of 69%.

The object of the invention was to provide a process with which biphenyl-2,2′-diols may be prepared with increased yield.

The object is achieved by a process according to claim 1.

Process comprising the process steps of:

a) adding sodium n-dodecyl sulfate,

b) adding a base,

c) adding a phenol of the formula (I):

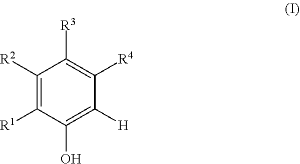

where R¹, R², R³, R⁴ are selected from:

—H, —O—(C₁-C₁₂)-alkyl, —(C₆-C₂₀)-aryl, —O—(C₈-C₂₀)-aryl;

wherein the alkyl groups and aryl groups cited may be substituted as follows:

substituted —(C₁-C₁₂)-alkyl groups, substituted —O—(C₁-C₁₂)-alkyl, substituted —(C₅-C₂₀)-aryl, substituted —O—(C₆-C₂₉)-aryl may have one or more substituents; the substituents are each independently selected from: —(C₁-C₁₂)-alkyl, —O—(C₁-C₁₂)-alkyl, halogen,

d) heating the reaction mixture of a) to c),

e) adding hydrogen peroxide to the reaction mixture,

f) cooling the reaction mixture so that the product (II) precipitates:

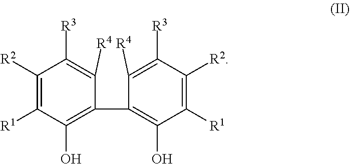

In one variant of the process. R¹, R², R³, R⁴ are selected from;

—H, —(C₁-C₁₂)-alkyl, —O—(C₁-C₁₂)-alkyl.

In one variant of the process, R² and R⁴ are each —H.

In one variant of the process, R¹ and R³ are selected from:

—(C₁-C₁₂)-alkyl, —O—(C₁-012)-alkyl.

In one variant of the process, R¹ and R³ are —(C₁-C₁₂)-alkyl.

In one variant of the process, the phenol (I) has the structure (3):

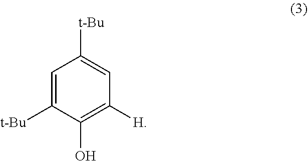

In one variant of the process, the base in process step b) is sodium hydroxide.

In one variant of the process, the heating in process step d) is carried out at a temperature in the range from 50° C. to 100° C.

In one variant of the process, the heating in process step d) is carried out at a temperature in the range from 65° C. to 95° C.

In one variant of the process, the addition of hydrogen peroxide in process step e) is carried out by dropwise addition of a hydrogen peroxide solution.

In one variant of the process, the temperature set in process step d) is maintained during the addition of hydrogen peroxide.

In one variant of the process, the temperature set in process step d) is maintained over a period in the range from 10 minutes to 2 hours after the addition of hydrogen peroxide.

In one variant of the process, this process comprises the additional process step f2):

f2) active cooling of the reaction mixture.

In one variant of the process, the active cooling is effected with the aid of an ice bath.

In one variant of the process, this process comprises the additional process step g):

g) washing the precipitated product with water.

The invention is to be illustrated in detail hereinafter by a working example.

### Synthesis of 3,3′,6,6-tetra-tert-butylbiphenyl-2,2′-diol (2)

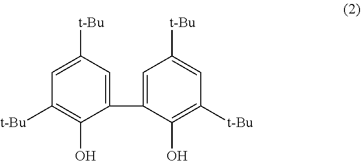 [SMILES: CC(C)(C)c1cc(-c2cc(C(C)(C)C)cc(C(C)(C)C)c2O)c(O)c(C(C)(C)C)c1]

To a solution of 1.2 g (4 mmol) of sodium n-dodecyl sulfate in 400 ml of demineralized water are added successively 64 g (1.6 mol) of sodium hydroxide and 166.72 g (0.8 mol) of 2,4-di-tert-butylphenol (3) with stirring. Subsequently, the solution is heated to 85° C. Then, over a period of 90 minutes, 58.4 ml (0.674 mol) of a 35% hydrogen peroxide solution are added dropwise, the reaction temperature being maintained between 85° C. and 90° C. This was achieved by lowering the bath temperature to 70° C. since the reaction is exothermic. After the dropwise addition, the mixture is stirred for a further 30 minutes at 85° C. Subsequently, the oil bath was set to 22° C. and the reaction solution cooled for ca. 2 hours. Subsequently, the reaction solution is placed in an ice bath for a further 1 hour. The reaction solution was then filtered and the residue was washed twice with 40 ml of water. The resulting solid was dried for 18 hours in vacuo. The yield was 166.3 g (99.2%).

The experiment conducted demonstrates that the object set is achieved by the process according to the invention.

1. Process comprising the process steps of:
a) adding sodium n-dodecyl sulfate,
b) adding a base,
c) adding a phenol of the formula (I): 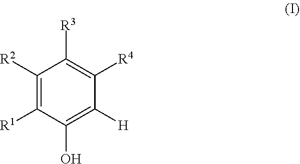
where R¹, R², R³, R⁴ are selected from:
—H, —(C₁-C₁₂)-alkyl, —O—(C₁-C₁₂)-alkyl, —(C₆-C₂₀)-aryl, —O—(C₆-C₂₀)-aryl:
wherein the alkyl groups and aryl groups cited may be substituted as follows:
substituted —(C₁-C₁₂)-alkyl groups, substituted —O—(C₁-C₁₂)-alkyl, substituted —(C₆-C₂₀)-aryl, substituted —O—(C₆-C₂₀)-aryl may have one or more substituents; the substituents are each independently selected from: —(C₁-C₁₂)-alkyl, —O—(C₁-C₁₂)-alkyl, halogen,
d) heating the reaction mixture of a) to c),
e) adding hydrogen peroxide to the reaction mixture,
f) cooling the reaction mixture so that the product (II) precipitates:

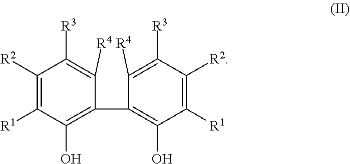

2. Process according to claim 1,
where R¹, R², R³, R⁴ are selected from:
—H, —(C₁-C₁₂)-alkyl, —O—(C₁-C₁₂)-alkyl.

3. Process according to claim 1, where R² and R⁴ are each —H.

4. Process according to claim 1, where R¹ and R³ are selected from:
—(C₁-C₁₂)-alkyl, —O—(C₁-C₁₂)-alkyl.

5. Process according to claim 1, where R¹ and R³ are —(C₁-C₁₂)-alkyl.

6. Process according to claim 1, wherein the phenol (I) has the structure (3):

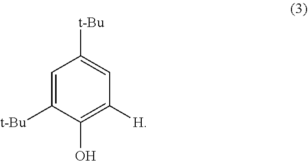

7. Process according to claim 1, wherein the base in process step b) is sodium hydroxide.

8. Process according to claim 1, wherein the heating in process step d) is carried out at a temperature in the range from 50° C. to 100° C.

9. Process according to claim 1, wherein the addition of hydrogen peroxide in process step e) is carried out by dropwise addition of a hydrogen peroxide solution.

10. Process according to claim 1, wherein the temperature set in process step d) is maintained during the addition of hydrogen peroxide.

11. Process according to claim 1, wherein the temperature set in process step d) is maintained over a period in the range from 10 minutes to 2 hours after the addition of hydrogen peroxide.

12. Process according to claim 1, comprising the additional process step f2):
f2) active cooling of the reaction mixture.

13. Process according to claim 12,
wherein the active cooling is effected with the aid of an ice bath.

14. Process according to claim 1, comprising the additional process step g):
g) washing the precipitated product with water.
